(19) United States
(12) Patent Application Publication
(10) Pub. No.: US 2008/0033371 A1
(43) Pub. Date: Feb. 7, 2008

(54) Cover for catheter assembly

(76) Inventors: Debra Kay Updegraff, Pleasanton, CA (US); Anders Ringheim Gjerde, Pleasanton, CA (US); Michael Edwin Serrano, Pleasanton, CA (US); Michael Patrick O'Neil, Pleasanton, CA (US)

Correspondence Address: Neil D. Gershon, 1011 High Ridge Road, Stamford, CT, 06905, US

(21) Appl. No.: 11/820,873

(22) Filed: Jun. 21, 2007

Related U.S. Application Data
(60) Provisional application No. 60/816,678, filed on Jun. 26, 2006.

(51) Int. Cl. A61M 25/00

(52) U.S. Cl. 604/263

(57) ABSTRACT

A cover for a proximal end of a connector attached to a catheter to receive an injection device. The cover comprises a body portion configured and dimensioned to receive at least a portion of an outer surface of the connector, a securement portion providing an engagement force on the connector for securing the cover to the connector, a removable cap having an internal portion with an anti-microbial agent, and a flexible member connecting the removable cap to the body portion.

This application claims priority from provisional application Ser. No. 60/816,678 filed Jun. 26, 2006, the entire contents of which is incorporated herein by reference.

### BACKGROUND OF THE INVENTION

1. Technical Field

This application relates to a catheter cover and more particularly to a resealable catheter cover having an antimicrobial agent.

2. Background of Related Art

Hospitals and health care providers are under increasing pressure to reduce costs while also improving patient care and safety. With the growing shortage of nurses in the United States, efficiency and quality in bedside care is becoming more critical.

Currently, the need for catheters for injecting medication is growing. With the growing use of catheters, the bloodstream infection rate from use of these catheters is also unfortunately growing. Many of these infections are caused by inadequate efforts to maintain an aseptic environment at the injection port for the catheter.

To facilitate medication injection, connectors have been developed and utilized with catheters. These connectors are attached at their distal end to the proximal end of the catheter tube via a luer lock. The proximal end of these connectors is configured to receive a syringe. One example of this type of connector is a positive pressure connector, such as the Maxplus. A syringe of medication can be luered within the proximal end of the connector and a spring inside forces fluid through the catheter when the syringe is removed, thereby reducing catheter occlusions. To maintain an aseptic environment, the input port of the connector receiving the syringe is recommended to be scrubbed with alcohol for thirty seconds before syringe attachment. This process to create an aseptic environment, however, in actuality is not commonly followed, especially in time critical situations such as in Intensive Care Units or in emergency situations. It is estimated by some that scrubbing time by nursing staff is typically as little as a few seconds, or not at all, i.e. inadequate to maintain asepsis.

Moreover, the connector, when not in use, will oftentimes rest on the patient's skin or bedding, thereby leaving the syringe receiving end in contact with non-sterile objects, increasing the necessity for proper disinfection.

As a result of the above described failure to adequately ensure the input port of the connector is disinfected prior to use, bloodstream infections in the hospital occur frequently. These infections not only prolong hospital stay, increasing costs, but can cause serious complications and even result in patient death. The risk is even greater due to the fact that the majority of the patients that have catheters for treatment are immuno-compromised with a reduced ability to fight such infections.

Therefore, the need exists to ensure an aseptic environment at the entry ports for catheter connectors. That is, it would be advantageous to provide a device that is simple to use, that when closed protects the end of the catheter, and when open provides access to an aseptic connector. This will eliminate the need for scrubbing of the connector prior to attaching a syringe, and thereby reduce the risk of infection. Such a device that can be opened quickly with a single hand of a user would also be advantageous.

### SUMMARY OF THE INVENTION

The present invention advantageously provides a device to maintain an aseptic environment at the fluid injection or extraction port for injection of medication or other fluids (or extraction of fluids) through a catheter. The present invention provides a cover for a proximal end of a connector which is attached to a proximal end of the catheter to receive a fluid transfer device, i.e. a fluid injection and/or extraction device. The cover comprises a body portion configured and dimensioned to receive a portion of an outer surface of the connector, a securement portion providing an engagement force on the connector for securing the cover to the connector, and a removable cap. A flexible member connects the removable cap to the body portion and the cap is manually movable by a user between an open position exposing an input port of the connector and the closed position covering the input port of the connector. An antimicrobial agent in an internal portion of the cap comes into contact with the input port of the connector when the cap is moved to the closed position to maintain an aseptic environment.

In one embodiment, the flexible member connecting the cap is integral with the body portion. In another embodiment, the flexible member is removably mounted to the body portion. In a preferred embodiment, the cap is connected such that it can be moved between the open and closed positions with a single hand of the user, and includes a thumb tab.

The body portion preferably forms a cradle having a concave surface to receive an outer portion of a cylindrical portion of the connector.

Preferably, the securement portion comprises a strap extending transversely to the body portion. The strap in one embodiment comprises a flexible member having a hook at one end to engage a ledge on the body portion as the strap extends around a circumferential portion of the connector.

In a preferred embodiment, the cap has a diaphragm on the internal portion with the anti-microbial agent shielded by the diaphragm such that the input port of the connector penetrates the diaphragm and contacts the agent when the cap is attached to the input port. The cap is preferably snap fitted onto the input port of the connector.

The present invention also provides a cover for creating and maintaining an aseptic environment at the proximal end of a connector attached to a catheter. The cover comprises a base portion having a region for receiving at least a portion of an outer surface of the connector, a securement portion configured to provide a frictional force to secure the cover to the connector, a removable cap hingedly connected to the base portion, and a diaphragm on the internal portion of the cap for maintaining an antimicrobial environment within the cap, wherein the antimicrobial agent is transferable to a portion of the connector upon attachment of the cap to the connector.

The present invention also provides a method for maintaining an aseptic environment for mounting of a fluid injection or extraction device to a connector which has been mounted to a catheter. The method includes providing a cover having a body portion and a cap connected thereto by a flexible member and having an antimicrobial agent on an internal surface, securing the body portion of the cover to the connector so the connector is seated within a receiving area of the body portion, placing the cap on the input port of the connector so the agent of the cap comes into contact with the input port, and thereafter removing the cap and mounting the fluid injection or extraction device to the connector.

The method may further comprise the step of maintaining an aseptic environment within the cap. This is achieved in one embodiment by a diaphragm in the cap. In a preferred embodiment, the steps of placing the cap and removing the cap can be performed with a single hand.

### BRIEF DESCRIPTION OF THE DRAWINGS

Preferred embodiment(s) of the present invention are described herein with reference to the drawings wherein:

FIG. 1 is a perspective view of a first embodiment of the cover of the present invention shown mounted to a conventional connector for a catheter, the cap shown in the open position;

FIG. 2 is an exploded view of the cover and connector of FIG. 1;

FIG. 2A is a perspective view of an alternate embodiment of the cap having an internal thread;

FIG. 3 is a side view in partial cross-section of the cover and connector taken along line 3-3 of FIG. 1;

FIG. 3A is an enlarged cross-sectional view of the cap of FIG. 1;

FIG. 3B is an enlarged cross-sectional view of the cap of FIG. 2A

FIG. 4A is a close up view of the attachment region of the retaining strap and cover of FIG. 1;

FIG. 4B is a close up view of an alternate embodiment having a removable strap and showing the region of the strap connecting to the cover;

FIG. 5 is a view similar to FIG. 3 except showing the cap in the closed position over the input port of the connector;

FIG. 5A is a close-up view of the area of detail of FIG. 5;

FIG. 6 is a perspective view of another alternate embodiment of the cover of the present invention;

FIG. 7 is a perspective view of yet another alternate embodiment of the cover of the present invention having a cap removable from the base of the cover;

FIG. 7A is a perspective view of another alternate embodiment of the present invention having a cap removable from the base of the cover; and

FIG. 8 is a cross-sectional view of an alternate embodiment of the cover.

### DETAILED DESCRIPTION OF PREFERRED EMBODIMENTS

There are many types of catheters for fluid transfer (injection and/or extraction) from patients such as peripheral IV catheters, peripherally inserted central line catheters, non-tunneled central line catheters, tunneled central line catheters, arterial line catheters, hemodialysis catheters, CVVH catheters, and epidural catheters. All these catheters are placed with the intent of being accessed repeatedly over a period of time.

Currently, connectors are attached to the proximal end of the catheter tube which extends from the patient. The proximal end of the connector has an input port to receive a syringe or other device. One example of this type of connector is the MaxPlus connector. The present invention provides a cover for these connectors, including the MaxPlus, which otherwise could provide a pathway for bacteria through the catheter and into the patient as syringes or other injection devices (or extraction devices) are repeatedly attached and removed from the connector. The cover of the present invention provides an aseptic environment at the point of access to the connector, namely the input port, and thereby an aseptic environment for fluid injection and/or extraction.

As described herein, the term “catheter assembly” includes a conventional catheter and a conventional connector, such as a positive pressure connector. Only the proximally extending tube of the catheter is illustrated in the drawings and designated as “T.” The connector shown in the drawings is a MaxPlus connector by way of example, designated by reference letter “C”, but it should be understood that the cover of the present invention can be used with other connectors, including other positive pressure connectors.

As used herein the term “proximal” refers to the portion further from the patient and the term “distal” refers to the portion closer to the patient.

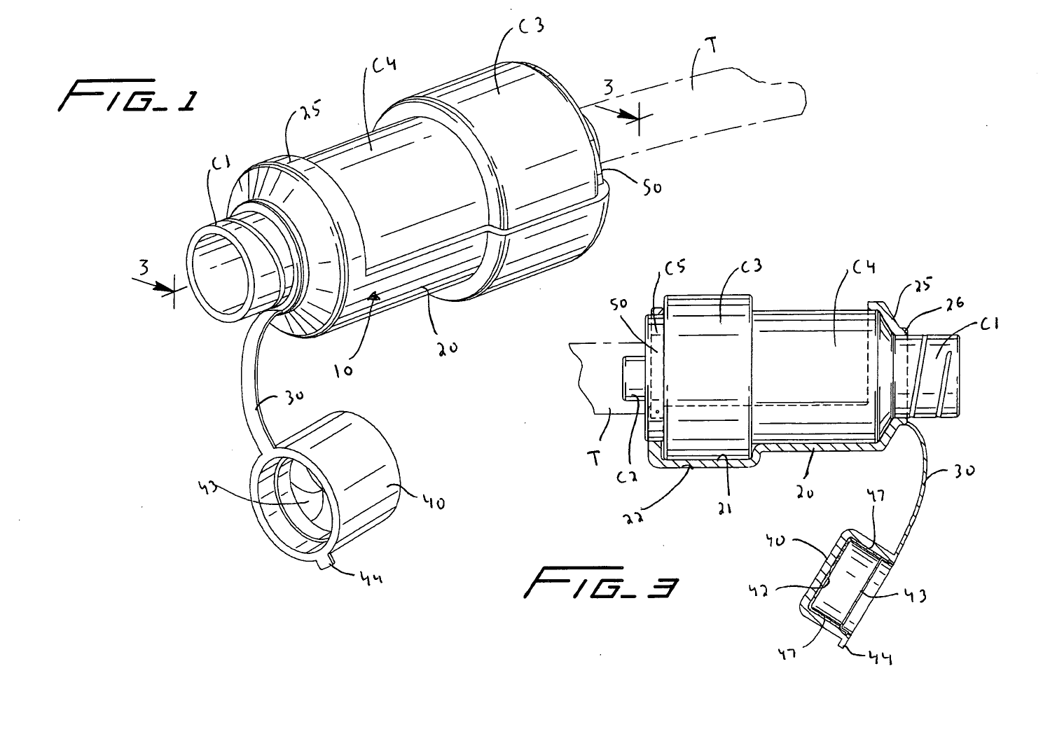

Referring now in detail to the drawings wherein like reference numerals identify similar or like components throughout the several views, FIGS. 1-3 illustrate a first embodiment of the cover of the present invention, designated generally by reference numeral 10. Cover 10 includes a mounting body portion 20, a hinge or tether 30, a cap 40 containing an antimicrobial agent 42, and a retaining strap 50. Cap 40 is attached to the flexible hinge 30 and is manually movable between open and closed positions as described in more detail below. The cover is preferably made of a light weight thin polymeric material, although other materials are contemplated.

The cover 10 is attached to the conventional connector C which connects to a proximal end of the catheter. Connector C has a tubular portion C2 extending from its distal end which has a threaded portion to threadedly connect to the proximal end of the catheter tube T. (The remaining portions of the conventional catheter are not shown as the catheter itself is not part of the present invention). The connector C is cylindrically shaped, with various diameter cylindrical portions: a large diameter region C3 at the distal end, a smaller diameter region C4 at an intermediate portion, and an even smaller diameter region C1 at the proximal end which has a threaded portion as shown to provide an input port to receive a syringe.

Referring back to the cover 10 of the present invention, the body portion 20 has a distal end 22 and a proximal end 24 and forms a cradle 21. The cradle has an inner concave surface 23 which preferably is stepped to substantially conform to the shape of the catheter connector C (e.g. regions C4 and C3). The cradle 21 preferably extends around 180° degrees of the connector, but it could also extend a shorter or greater amount around the connector. The bottom surface of the cradle 21 helps to raise the connecting portion C1 of connector C from a non-sterile surface. That is, the cradle 21 will further space portion C1 from the non-sterile surface, thereby reducing the chances of contact with the surface. The cradle can alternatively have a substantially planar lower surface to better enable it to maintain a set position when placed on the patient's skin or bed, further reducing the chance of unwanted contact with a non-sterile surface.

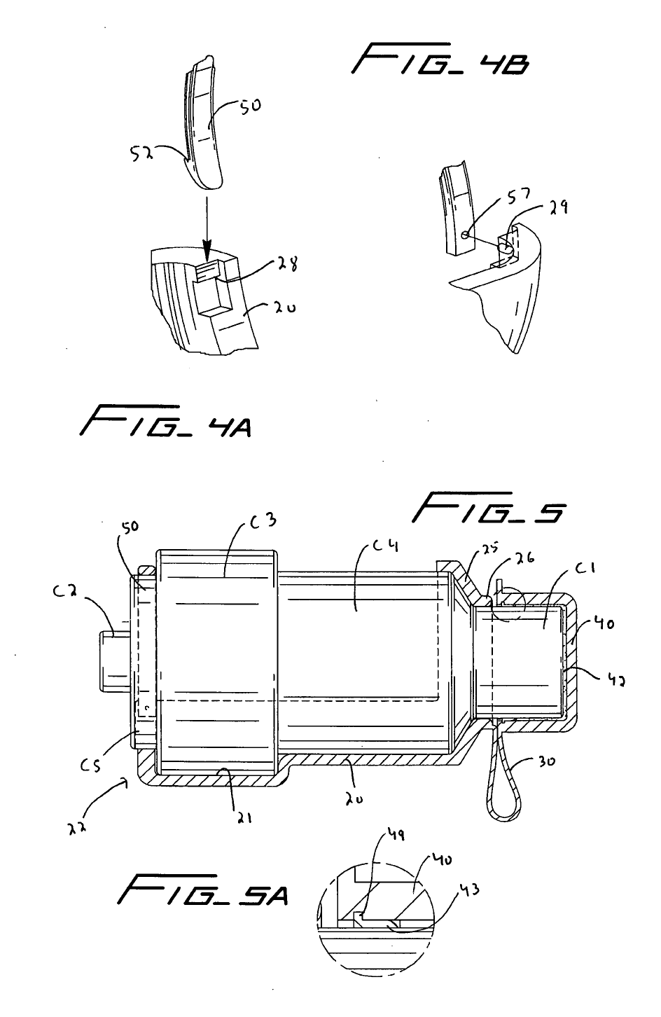

The distal end 22 of cover 10 has a securement portion or securement member in the form of a retention strap 50 which is positioned transverse to the body portion and extends around the circumference of the connector. More specifically, strap 50 has a hook 52 at one end which engages a ledge 28 on body portion 20 (see also FIG. 4A). For mounting to the connector, strap 50 wraps around the circumference of the connector, i.e. portion C5, and the hook 52 is engaged with the ledge 28 to apply a circumferential engagement force against the connector for securement thereto.

The strap 50 can be removably mounted to the cover to enable straps of different sizes to be used. One way to removably mount the strap is shown in FIG. 4B where pin 29 on the body engages aperture 57 on the strap. This could accommodate connectors of different sizes by using longer or shorter straps to conform to larger or smaller diameters of various connectors. It is also contemplated that the strap 50 could be adjustable so that the hooks at one end could engage one of various ledges or openings of the body portion to accommodate different diameters. A ratchet type mechanism is also contemplated. The ledge or opening could alternatively be placed on the strap with the hook end overlapping (extending more than 360°) for engagement.

The proximal end 24 of the body 20 includes a ring like portion 25 with an O-ring lip 26 having an opening 27. When mounted to the connector C, the portion C1 of connector C extends through the opening 27 and the ring portion 25 extends around a circumference of the connector C as shown. It should be appreciated that alternatively the ring portion could extend around less than 360 degrees, e.g., being C-shaped to surround less than 360 degrees of the outer surface of the connector C.

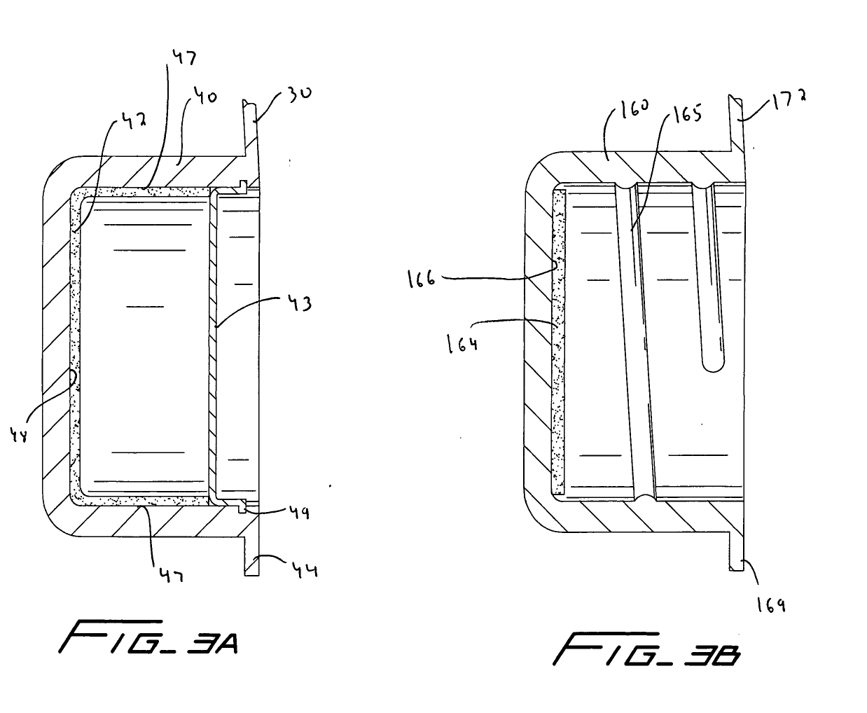

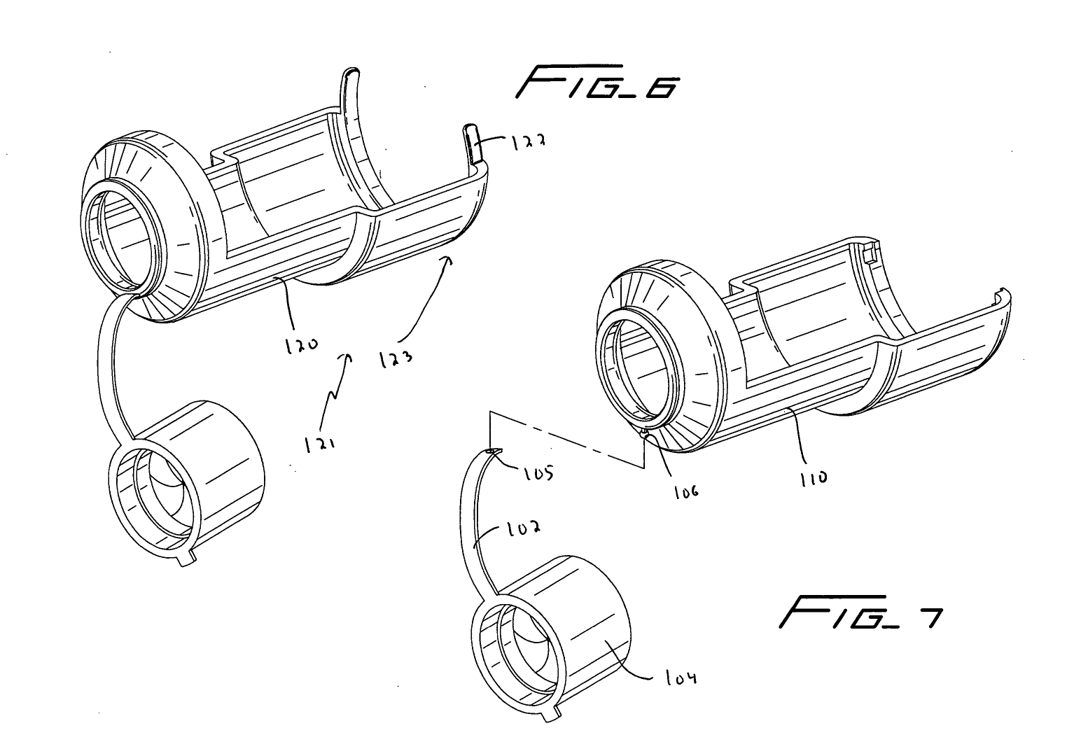

Flexible hinge 30, which can be in the form of a strap or a thin material, allows for movement of the cap 40 from an open position as shown for example in FIG. 3 to a closed covering position where it covers at least a portion of region C1 of connector C as shown in FIG. 5. (The catheter tube has been removed from this Figure for clarity).

The cap in one embodiment has an internal lining containing an antimicrobial agent. Various agents can be utilized. One agent which can be used, for example, is chlorhexidine gluconate, which is currently used to prep the patient's skin before surgery and has the advantage of lasting several days. Another agent which can be used, by way of example, is silver iodine.

The agent is in concentrations sufficient to maintain an aseptic environment for a period of time. The agent utilized can be liquid or non-liquid. It can be impregnated on a porous polymer internal portion of the cap. Also, the agent could be suspended in a layer of synthetic biopolymer composite foam impregnated with the antimicrobial agent.

In a preferred embodiment, the cap 40 includes a diaphragm 43 (see e.g. FIGS. 1 and 3A) which provides a barrier or shield for the agent 42 (disposed proximal of the diaphragm) to create a closed environment inside the cap and to separate the agent 42 from the external environment. The diaphragm 43 has a lip 49 to engage a recess in the cap wall (see also FIG. 5A), to hold the diaphragm 43 in the cap 40 during insertion and removal of connector portion C1 through the diaphragm 43. The agent 42 can be on the proximal surface 48 of the cap 40 as well as the sides 47 (e.g. a circumferential area distal of surface 48) as shown. When the cap 40 is attached to the portion C1 of connector C, portion C1 penetrates the diaphragm 43 and comes into contact with the agent. When removed, the diaphragm automatically seals to confine the agent. Thus, as can be appreciated, the cap forms both a housing and a delivery mechanism for antimicrobial material to the medical access port.

The cap can be repeatedly placed on and then taken off as needed for access to the catheter via the connector thereby maintaining an aseptic environment for multiple injections or extractions. That is, when the catheter is not being used for injection (or extraction), the cap 40 is snap fit over the portion C1 of connector C to the closed position which enables contact of the connector portion C1 with the agent 42. Consequently, when the cap 40 is removed (to the open position) in order to attach a syringe or other fluid injection device to the input port of portion C1, the agent 42 maintains an aseptic environment to prevent entry of bacteria.

The cap can also be quickly removed and attached with one hand, thereby freeing the user's other hand. Thumb tab 44 aids in removal as the user can apply pressure to the tab with his thumb to overcome the frictional engagement (or snap-fit) of cap 40 with connector portion C1. Preferably, the cap can also be placed on the connector portion C1 with one hand as well.

Cap 40 can be made of a variety of materials. In one embodiment, it is made of an ultra-thin light weight polymer such as polypropylene.

The cap preferably snaps onto the tubular portion C1 for easy one-handed operation. Some of the agent would automatically dislodge from the cap lining by the frictional engagement or interference fit of the cap with the tubular portion C1. As an alternate, the cap can snap fit onto a portion of the cover itself. This is shown in the embodiment of FIG. 8 wherein cap 140 is snap fit over distal portion 144 of cover 142, engaging circumferential raised surface 147. This provides a self contained unit in that the cap relies on a portion of the cover for attachment instead of a portion of the connector. Connector portion C1 penetrates diaphragm 148 for contact with agent 149. Other structure can be utilized to connect the cap to portion C1, such as a thread design as discussed below.

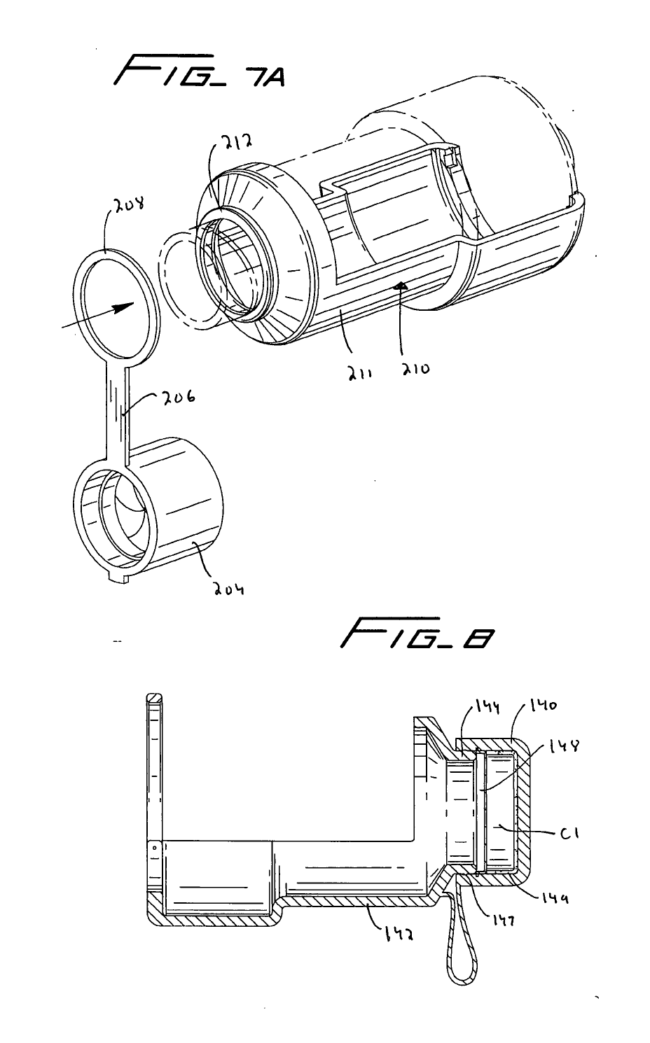

Additionally, although hinge or tether 30 is shown integral with the body 20, it is also contemplated that the hinge (and attached cap) can be removably mounted to the cradle. One example of a removable cap and hinge is shown in FIG. 7, wherein the cap 104 is attached to the tether 102, and the tether 102 is removable from the body portion 110. That is, a pin 106 or other structure, can be attached to the connector C and configured to mate via a snap fit with the aperture 105 in tether 102. The removable cap 104 enables a cap with a full amount of antimicrobial agent to replace a cap which has less agent after repeated on and off engagement with the connector C. The cap could alternately be removably mounted to the tether.

Another example of a removable cap and hinge is shown in FIG. 7A. Closure assembly includes cap 204, hinge or tether 206 and O-ring 208. O-ring 208 removably mounts for frictional engagement to lip 212 of body portion 211 of cover 210. Otherwise, cap 202 and body portion 211 (and cap 104 and body portion 110) are substantially identical to cover 10 of FIG. 1.

It is also contemplated that in an alternate embodiment, ring 208 can be permanently attached to the cover to provide a single unit.

In an alternate embodiment of FIG. 6, cover 121 is similar to cover 10 of FIG. 1 except for the C-shaped tensioning clamp portion 122. That is, the distal end 123 of body portion 120 has a forked member, preferably C or U-shaped, which forms a tension clamp to secure the cover 120 to the connector. Clamp 122 applies a force against the large diameter cylindrical portion C3 of connector C (not shown) for frictional securement of connector C.

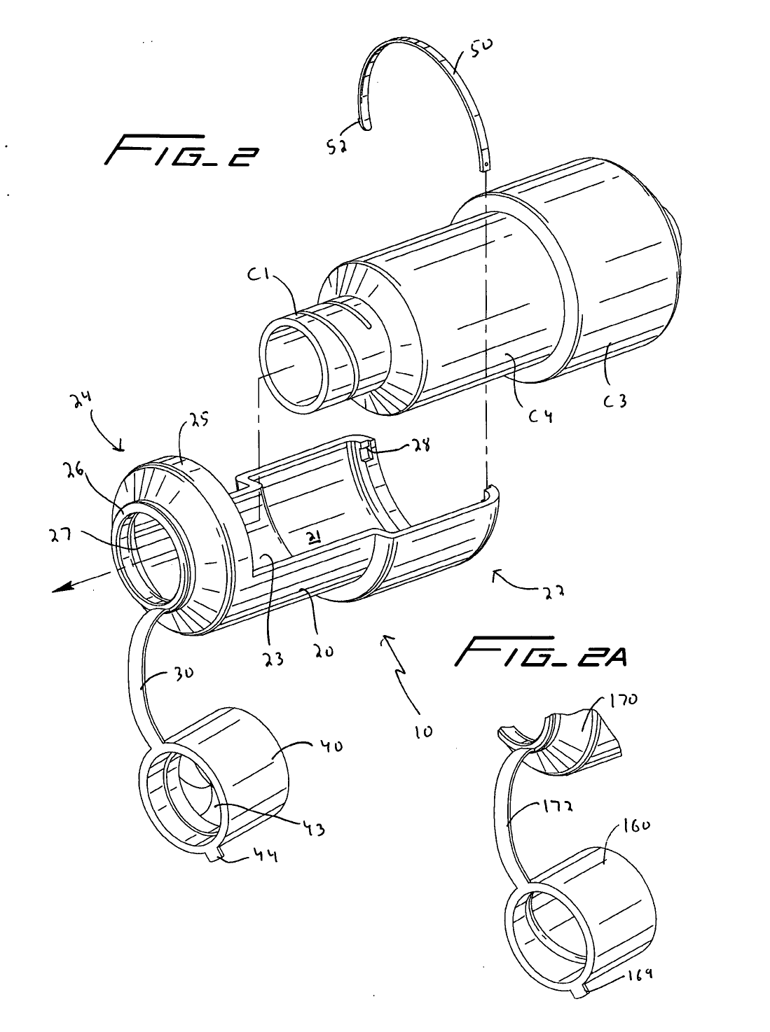

In an alternate embodiment which may require two handed operation, but still provide the advantage of maintaining an aseptic environment, the cap would rotate onto the connector. In this version, shown in FIGS. 2A and 3B, the rotation of the cap 160 would aggressively dislodge the antimicrobial agent onto the tubular portion. Slight rotation would be preferable so as not to unduly twist the tether. In this embodiment, anti-microbial agent 164 is disposed on the proximal wall 166 of cap 160, proximal of threads of 165. Cap 160 includes thumb pad 169 and is attached to cover 170 (only a portion of which is shown in FIG. 2A) by flexible member 172.

In use, after attachment of the connector to the catheter, and before or after initial use of the catheter, depending on the circumstances, the cover of the present invention is placed over the connector portion C1. The connector portion C1 penetrates the diaphragm and comes into contact with the antimicrobial agent on the internal portion of the cap. The agent contacts the end portion of the connector as well as areas around the circumference as the agent is on the back wall and side wall of the cap. This maintains an aseptic environment. If the connector is placed on non-sterile objects, such as the patient's skin or bedding, an aseptic environment is maintained.

When access to the catheter is needed for fluid injection (or extraction), the user can hold the cap or cover with his fingers and place his/her thumb on the thumb pad, and apply a proximal force to release the cap from the cover. This can be achieved with one hand, leaving the other hand free for other uses, such as access to other instrumentation. Since the agent is on the connector portion C1, when the cap is removed, an aseptic environment is maintained as the syringe is attached. Cleansing the connector portion or other action by the user to maintain an aseptic environment is not required. After attachment of the syringe or other fluid transfer device, and performance of the procedure, the fluid transfer device is removed, and the cap can be placed on the connector portion, preferably with one hand. Placement of the cap once again causes the diaphragm to be penetrated and the agent to come in contact with the connector portion. When additional treatment is required, the cap can be removed in the same manner as discussed above. The placement and removal of the cap can be repeated any number of times as needed, maintaining the aseptic environment. The diaphragm maintains the shielded environment within the internal portion of the cap.

In the aforedescribed embodiments having a replaceable cap, if additional agent is required, or a different agent is required, or a cap with a different structure for attaching to the connector portion, the used cap can be removed and replaced with a fresh cap without having to change the whole cover.

It is also contemplated that the cover of the present invention could alternately be placed directly on a catheter hub or other portion of the catheter rather than on a connector. The cradle would be modified to conform to the outer shape of the respective catheter portion.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims appended hereto.

What is claimed is:

1. A cover for a proximal end of a catheter assembly having a catheter extending into a patient and a connector attached to the catheter to receive an injection or extraction device, the cover comprising:
a body portion configured and dimensioned to receive at least a portion of an outer surface of the connector,
a securement portion, the securement portion providing an engagement force on the connector for securing the cover to the connector;
a removable cap having an internal portion, the internal portion having an anti-microbial agent, the agent coming into contact with an input port of the connector when the cap is moved to a closed position to create and maintain an aseptic environment; and
a flexible member connecting the removable cap to the body portion, the cap manually movable by a user between an open position exposing the input port of the connector and the closed position covering the input port of the connector.

2. The cover of claim 1, wherein the flexible member is integral with the body portion.

3. The cover of claim 1, wherein the flexible member is removably mounted to the body portion.

4. The cover of claim 1, wherein the body portion forms a cradle having a concave surface to receive an outer portion of a cylindrical portion of the connector.

5. The cover of claim 1, wherein the securement portion includes a C-shaped member.

6. The cover of claim 1, wherein the securement portion comprises a strap extending transversely to the body portion.

7. The cover of claim 6, wherein the strap comprises a flexible member having a hook at one end to engage a ledge, the strap extending around a circumferential portion of the connector.

8. The cover of claim 7, wherein the ledge is disposed on the body portion.

9. The cover of claim 1, wherein the anti-microbial agent is chlorhexidine gluconate.

10. The cover of claim 1, wherein the cap further comprises a thumb tab extending therefrom.

11. The cover of claim 1, wherein the cap has a diaphragm on the internal portion and the agent is proximal of the diaphragm, the input port of the connector penetrating the diaphragm and contacting the agent when the cap is attached to the input port.

12. The cover of claim 1, wherein the cap is snap fitted onto the input port of the connector.

13. The cover of claim 12, wherein the body portion forms a cradle having a concave surface to receive an outer portion of a cylindrical portion of the connector.

14. The cover of claim 1, wherein the cap is connected to the body portion such that it is fitted onto the input port and removed from the input port with a single hand of the user.

15. A cover for maintaining an aseptic environment at the proximal end of a connector attached to a catheter for fluid injection or extraction, the cover comprising:
a base portion having a region for receiving at least a portion of an outer surface of the connector;
a securement portion configured to provide a frictional force to secure the cover to the connector;
a removable cap hingedly connected to the base portion, the cap having an internal portion and an external portion and an antimicrobial agent within the cap; and
a diaphragm on the cap for maintaining an antimicrobial environment within the cap, the antimicrobial agent transferable to a portion of the connector upon attachment of the cap to the connector.

16. A method for maintaining an aseptic environment during mounting of a fluid injection or extraction device to a connector which is mounted to a catheter, the method comprising:
providing a cover having a body portion and a cap connected thereto by a flexible member, the cap having an antimicrobial agent on an internal portion;
securing the body portion of the cover to the connector so the connector is seated within a receiving area of the body portion;
placing the cap on an input port of the connector so the agent of the cap comes into contact with the input port; and
thereafter removing the cap and mounting the fluid injection or extraction device to the connector.

17. The method of claim 16, further comprising the step of securing a strap around an outer surface of the connector.

18. The method of claim 16, further comprising the step of maintaining an aseptic environment within the cap

19. The method of claim 16, wherein the step of removing the cap can be performed with a single hand.

20. The method of claim 19, wherein the step of placing the cap can be performed with a single hand.
